(19) United States
(12) Patent Application Publication
(10) Pub. No.: US 2010/0022912 A1
(43) Pub. Date: Jan. 28, 2010

(54) METHOD AND APPARATUS TO DETERMINE A PARAMETER PERTAINING TO THE PROSTATE OF A PATIENT

(76) Inventors: Jens Fehre, Hausen (DE); Ralf Nanke, Neunkirchen am Brand (DE); Martin Stetter, Muenchen (DE)

Correspondence Address: SCHIFF HARDIN, LLP;PATENT DEPARTMENT, 233 S. Wacker Drive-Suite 6600, CHICAGO, IL, 60606-6473, US

(21) Appl. No.: 12/508,723

(22) Filed: Jul. 24, 2009

(30) Foreign Application Priority Data
Jul. 25, 2008 (DE) 10 2008 034 703.5

(51) Int. Cl. A61B 10/00; A61B 5/00; G06Q 50/00

(52) U.S. Cl. 600/562; 600/300; 705/3

(57) ABSTRACT

In a method to determine at least one parameter pertaining to a prostate of a patient, an integrated diagnostic support system implements the following steps. A first risk value for prostate illness is determined using a screening method. In the event that this first characteristic risk value exceeds a first limit value, at least one second characteristic risk value for prostate illness and its location are determined using a method supplying a medical image of the patient. An integrated report system outputs at least one of the characteristic risk values and/or the location. A corresponding device has a mini-laboratory analysis unit for the screening test, an intelligent decision support system for the first characteristic risk value, a first interface with an imaging system, an evaluation system for the medical image for the second characteristic risk value, and a report system with a user interface.

### BACKGROUND OF THE INVENTION

1. Field of the Invention

The present invention concerns a method and an apparatus to determine a parameter that pertains to the prostate of a patient.

2. Description of the Prior Art

A goal of medicine today is to treat complex illnesses of a patient with therapy in an optimally customized and prompt manner. For this purpose, it is necessary to detect the presence and the type of the illness as early and precisely as possible. This is particularly true for various prostate illnesses in men, including prostate cancer (the most common cancer type in men).

For prostate illnesses, an early and differential diagnosis should be conducted in as many potentially at-risk (for example older) men as possible in order to increase the chances of recovery and to simultaneously reduce costs of follow-up treatments. The diagnosis of prostate illnesses according to the procedure typical today, however, is a multi-stage and laborious process.

FIG. 3 shows the typical procedure in a prostate diagnosis today in a flowchart over time t. In a first visit 300a of a patient 302 to a physician's practice 304, a blood sample 306 is initially extracted from the patient 302. To determine a PSA value 308 (thus the concentration of the “prostate-specific antigen”) from the blood sample 306, this is sent to a central laboratory 310 that conducts the evaluation and sends the result to a physician 312 in the practice 304. The PSA value 308 is an indicator of prostate pathologies, but with limited specificity. This procedure extends over at least one day, often multiple days.

If this first examination (thus the visit 300a) is conducted by a physician in private practice and raises suspicion, the patient 302 is referred to a hospital 314 where the aforementioned PSA value 308 is typically determined again for confirmation, according to the aforementioned procedure in a visit 300b, possibly by a different central laboratory.

If the result of the PSA test confirms the suspicion of a prostate illness, the patient 302 is called back into the hospital 314 a second time for an appointment 300c, thus for TRUS (transrectal ultrasound) examination 316. The prostate 318 of the patient 302 is hereby locally searched for indications corroborating the suspicion. In many cases, however, the TRUS examination 316 is also conducted at the first visit 300b.

If the PSA or TRUS finding corroborates the suspicion of illness, the patient 302 is called back a second or third time for an appointment 300d to conduct a biopsy 320. Since it cannot be ensured that the biopsy needle 321 hits a possible focus of the illness (for example an inflammation or a tumor), such a biopsy 320 must sometimes be conducted multiple times.

Newer approaches to the biopsy 320 use a procedure known as a directed (guided) biopsy, i.e. an ultrasound imaging 322 with simultaneous contrast agent administration 324, in order to direct the biopsy needles 321 in a targeted manner to the disease focus. This reduces the physical and psychological stress for the patient 302 since normally only a single biopsy examination 320 must be conducted.

In total the standard procedure described in FIG. 3 therefore normally extends over multiple weeks and must be conducted in multiple sessions or visits 300a-d.

Parameters are thus surveyed or collected from the patient with the known method. The parameters are, for example, laboratory values, ultrasound or radioscopy image or other finding values. These parameters reflect the status of the prostate of a patient, and thus are also associated with a prostate illness of the patient. In other words, such parameters form the database for a physician who, using these, presents a diagnosis for the patient via his or her personal evaluation of the facts in order to exclude a prostate illness or to now diagnose it, for example according to its type and severity.

A medical examination unit with integrated mini-laboratory analysis unit is known from DE 10 2007 026 910 A1, published on 18 Dec. 2008. The unit, which can primarily be used in emergency rooms, intensive care units, OP rooms etc., contains at least one ultrasound, patient monitor, EKG, respiration and/or resuscitation (CPR) device for monitoring the vital functions and/or for emergency treatment of emergency patients by emergency doctors. Multiple medical examinations can be integrated using this examination unit, i.e. they can be conducted in one continuous (uninterrupted) workflow.

### SUMMARY OF THE INVENTION

An object of the present invention is to provide an improved method and a device with which the determination of a parameter pertaining to the prostate of a patent can be conducted.

The object with regard to the method is achieved by a method to determine at least one parameter pertaining to the prostate of a patient with the following steps tat are implemented by a device, i.e. an integrated diagnostic assistance system in an integrated workflow. A first characteristic risk value for the presence of a prostate illness is first determined for the patient using a screening method. In other words, the global risk (thus the risk as measured for the entire body) for the presence of a prostate illness is determined as the result of the screening test or, respectively, as a first characteristic risk value. For example, the established PSA level can be measured within the scope of the screening along with properties derived from this. A marker panel, i.e. multiple markers simultaneously, can also be used with higher sensitivity and specificity.

In the event that the first characteristic risk value exceeds a first limit value, a second characteristic risk value for the presence of the prostate illness and the suspected location of the prostate illness is determined using a method supplying a medical image (for example an ultrasound or radioscopy image) of the patient.

In this method step the risk for the existence of an illness that is locally measured at the specific patient is thus identified with a first, non-invasive method (using imaging methods, for example ultrasound) if there is an existing global risk (and only then). The imaging methods can if necessary be combined with non-specific or illness-specific in vivo markers, for example a contrast agent. Moreover, in the diagnostic step the medical image generated via the imaging method later provides the physician with more detailed information about the type of illness to be expected in the patient, for example the presence of prostatitis or prostate cancer.

After at least one of the conducted steps, an integrated report system outputs at least one of the values or data (thus one of the characteristic risk values and/or the location) that has been determined, or was already provided in the preceding method workflow as a parameter. In other words, all examinations, findings and risk assessments are displayed to the physician as a type of integrated report system in the manner of an overview supporting the final diagnosis. A quality standard of the results adduced by the physician is thus endured. Moreover, it is ensured that the method workflow ensues according to the level of the valid medical guidelines.

The display of such an integrated report ensues via a user interface, for example. The report presents a collected summary sheet of the diagnostic-therapeutic workflow. It shows the current data, image, analysis, finding and risk situation relevant to the diagnosis and therapy in a qualitatively certain manner according to medical guidelines.

The invention is based on providing suitable analysis and diagnostic methods in an integrated form that can be implemented by a physician in private practice. The process of the parameter collection (and therefore also the subsequent diagnosis generation of prostate illnesses by a physician) is thus accelerated and made more (cost-)efficient. The method can thereby be conducted on a broader group of potentially at-risk people that is to be combed through.

In other words, the method according to the invention describes an integrated solution for the preparation of a diagnosis and, if necessary, therapy or theranosis of prostate illnesses. All method steps are provided by the method in a manner such that, first, they can be conducted immediately and, second, they can respectively be conducted on site, for example all together at a clinic or by a physician in private practice.

Essential features of the method are therefore the identification and the customized combination of components or individual steps into a workflow that assists a physician (for example a urologist) in an optimal manner.

The method offers an optimized workflow for the urologist. The urologist can thereby ensure the quality of his or her service, increase the level of health care of his or her patients and reduce the costs of diagnosis by sparing unnecessary diagnostic methods.

The method and corresponding device can be modular in design and can be adapted to the demands of individual hospitals or, respectively, practices.

The method offers a significant time savings for the preparation of a diagnosis in comparison to the current state of the art. It reduces discomforts and psychological stresses for the patient. It can be adapted flexibly to new guidelines and be efficiently optimized, namely by replacing existing method modules with respective newer and improved method parts. An expansion of the purely diagnosis is possible, for example with a therapy preparation, for example via integrated planning or incorporation of the assistance in local therapies such as (for example) high-intensity focused ultrasound (HIFU).

In a preferred variant of the method, the following steps are additionally executed. In the event that, in addition to the aforementioned condition for the first limit value, the second characteristic risk value also exceeds a second limit value, a tissue status (thus a local illness status) is determined in the patient using an invasive method at the location of the prostate illness. The tissue status can then optionally also be output via the report system.

In this method step a second, invasive method is thus implemented only given an existing global risk and local risk—and only then. This allows parameters of the patient to be acquired that later allow definitive clarity about the tissue status and the type and severity of the illness of the patient to be acquired in the diagnosis by the physician.

In a preferred embodiment, the screening method is conducted with the incorporation of an in vitro test. This test is in turn implemented on site in a mini-laboratory analysis unit.

In an additional embodiment, the first characteristic risk value is determined using an IT-based decision support system.

In an alternative embodiment of the method, the second characteristic risk value is determined using prostate-specific variations. These are learned from the generated medical image.

In one variant of the method, an evaluation system (for example in the form of software) again calculates the second characteristic risk value. The local distribution of this characteristic value (thus of the local risk) can then also be determined, for example in the form of a risk map.

The evaluation software can evaluate the results of ultrasound examinations. The software can determine the local probability distribution for the presence of pathological tissue (for example from contrast-intensified ultrasound images), for example of cancer tissue. This distribution is then designated as a risk map.

In a preferred embodiment the results of the imaging method, possibly superimposed with the risk map, can be used in a precise manner in the implementation of the invasive method to guide biopsy needles in the invasive procedure, for example.

In the method the determination of first and second characteristic risk value can advantageously be conducted using a knowledge management system. For example, this consists of an integrated medical patient database and a web portal via which additional information related to the prostate theranosis can be retrieved in a context-sensitive manner.

In a preferred embodiment of the method, an integrated medical database is used that is directed either locally or centrally. The data records in the database allow the decision assistance in the method workflow to be optimized, for example the level of the limit values. The database can moreover be used for quality assurance.

In specific embodiments, the method therefore offers software assistance via a knowledge discovery and knowledge management system that allows the calculation of quantitative (and therefore reproducible) risk probabilities.

With regard to the device, the object is achieved by a device to determine at least one parameter pertaining to the prostate of a patient. For example, the core of the device is a workstation with various components: the device has a mini-laboratory analysis unit connected to the workstation, for example in the form of a lab-on-a-chip in vitro diagnostic unit to measure the PSA level or, respectively, composite serum marker. The mini-laboratory analysis unit therefore serves to conduct a screening test on a patient. The device moreover has at least one intelligent decision support system to evaluate the screening data. For example, a Bayesian network can hereby be used whose properties were learned from data about many patients, for example via data mining and knowledge discovery. The decision support system therefore serves to determine a first characteristic risk value for the presence of a prostate illness using the screening test.

The device has a first interface with an imaging system to produce a medical image of the patient, thus with an imaging apparatus. For example, an ultrasound apparatus (which can be used flexibly in comparison to an MR examination) can be used here.

The device also has an evaluation system to determine at least one second characteristic risk value for the presence of the prostate illness and its suspected location from the medical image; and a report system with a user interface to output at least one parameter, wherein the parameter is one of the characteristic risk values and/or the location.

The device according to the invention thus has a series of interfaces. These support a modular (and therefore highly flexible) design of the device.

The device and its advantages have otherwise already been explained in connection with the method according to the invention. Such a device is called a “ProstaStation”, for example, and represents an integrated solution that can be used for the diagnosis of prostate cancer, for example.

In a preferred embodiment, the device contains another second interface with an invasive prostate examination system delivering a tissue status of the patient at the location. The tissue status can then optionally also be output by the report system.

As mentioned, the device can have an interface with a knowledge management system that delivers parameters that pertain to prostate illnesses.

As likewise mentioned, the knowledge management system can comprise a medical patient database and an interface with the Internet. The decision support system can then be used in connection with this to calculate a global risk (thus a first characteristic risk value in the form of a probability) for a new patient on the basis of database contents and new patient data.

### BRIEF DESCRIPTION OF THE DRAWINGS

FIG. 1 shows the workflow according to the invention of a parameter determination on a prostate patient.

FIG. 2 sows the workflow from FIG. 1 in more detail.

FIG. 3 shows the workflow of the parameter determination according to the prior art.

### DESCRIPTION OF THE PREFERRED EMBODIMENTS

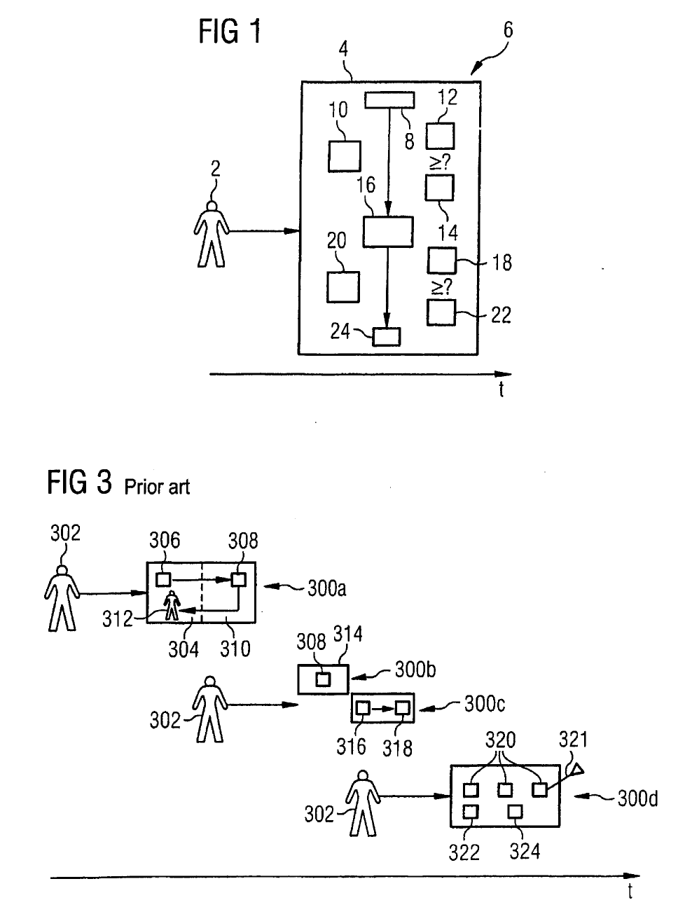

FIG. 1 shows a workflow over time t of an integrated data collection on a patient 2 for the purpose of diagnosing a prostate illness, in a generalized representation. The data collection is conducted in a medical practice 4 that the patient visits in a first appointment 6. An in vitro test 8 is initially conducted as a screening test. A decision support system 10 determines as a first characteristic risk value a global risk 12 that the patient 2 exhibits a prostate illness.

If the global risk 12 exceeds a first limit value 14, a TRUS examination 16 is conducted on the patient 2. A local risk 18 that the patient 2 suffers from a prostate illness is determined using this TRUS examination 16. An integrated finding report 20 about the TRUS examination 16 is simultaneously generated.

If the local risk 18 exceeds a second limit value 22, a biopsy 24 is conducted on the patient 2.

All steps shown in FIG. 1 are respectively immediately conducted in succession and on site in the medical practice 4, such that the entire method takes only a few hours.

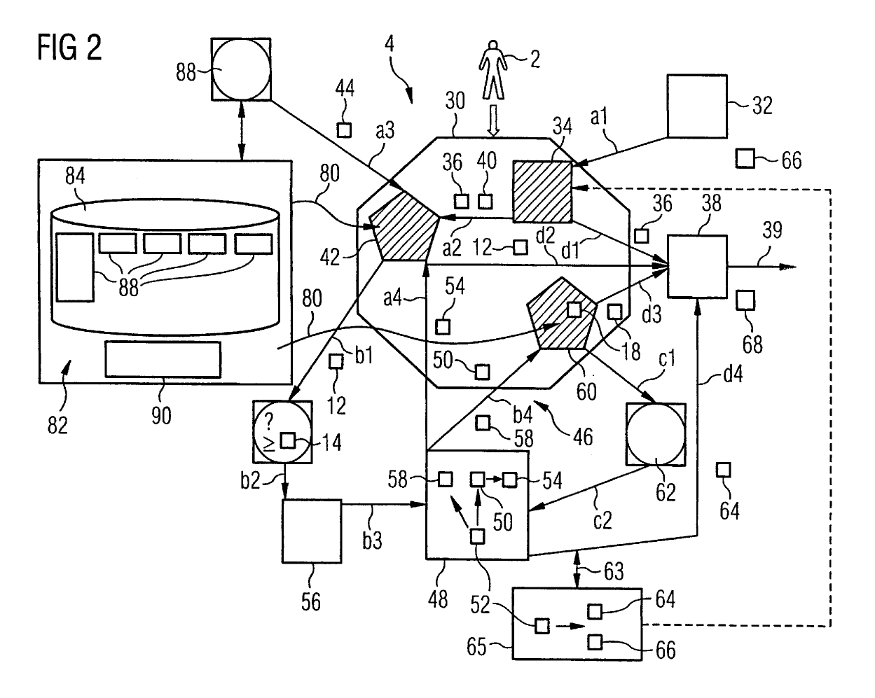

FIG. 2 shows the workflow of the method from FIG. 1 in detail. The patient 2 visits the medical practice 4 to clarify complaints, which medical practice 4 possesses a ProstaStation 30 as an integrated device for data collection. A general screening—designated with a)—is initially conducted on the patient. A blood and/or urine sample 32 is taken from the patient and supplied along the arrow a1 to an in vitro analysis unit 34 belonging to the ProstaStation 30.

The resulting concentration values 36 are then copied along the arrow d1 to a reporting system 38 for display. The reporting system has a user interface 39 at which all report results can be viewed by a physician (not shown). All procedures that belong to an integrated report are always designated with d). At the same time, they are relayed along the arrow a2 together with derived values 40 as an input to the downstream decision support system 10. This is a software system of the ProstaStation 30 and conducts decision support. The decision support system 10 automatically loads additional available data 44 of the patient 2 (for example anamnesis data such as age, weight, history or earlier examination results which can simultaneously serve as input for a risk evaluation) along the arrow a3.

An interface 46 with an ultrasound apparatus 48 is located at the ProstaStation 30. Finally, ultrasound exposures 50 of the prostate 52 of the patient 2 are generated from the patient 2 for primary clarification. Typical characteristic values 54, for example prostate volume or the presence of lesions, are determined from this and transmitted to the decision support system 10 along the arrow a4.

The decision support system 10 calculates a probability value for the global risk 12 on the basis of these screening data, for example the concentration values 36, which probability value is then transmitted along the arrow d2 to the reporting system 38 for display.

If the global risk along the arrow b2 exceeds the first limit value 14 as a threshold, or, respectively, if the physician optionally decides that a risk case is present, a more precise clarification of the local conditions in the prostate 52 immediately ensues along the arrow b2 in the form of a contrast-intensified ultrasound examination with the ultrasound apparatus 48. This first, non-invasive examination is generally designated with b).

For example, a contrast agent 56 marked with a molecular marker for prostate cancer, prostatitis or both with offset can thereby also be injected along the arrow b3. The resulting contrast images 58 are supplied along the arrow b4 to an analysis software 60. The analysis software 60 as part of the ProstaStation 30 determines from this the local risk 18 in the form of a risk map 62 and transmits this along the arrow d3 to the reporting system 38 for display.

Finally, if the local risk values 18 also exceed the second limit value 22 or if the physician decides that an increased risk is present, a second, invasive examination c) in the form of an invasive tissue extraction or, respectively, biopsy is conducted along the arrow c1. For this the ProstaStation 30 has an interface 63 with an invasive examination system 65. The contrast agent-intensified ultrasound examination b can hereby serve (along the arrow c2) for a targeted extraction in the examination system 65 in that the risk map 62 is used.

In the case of a real-time calculation of the risk map 62, one and the same ultrasound examination can in particular serve for a first examination b and the generation of the risk map 62 and to assist the second examination c (thus the biopsy). This leads to a significant increase of the efficiency of the entire method and a distinct reduction of the stress for the patient 2.

The result 64 of the biopsy is supplied to the reporting system 38 along the arrow d4. This result 64 can thereby originate from, for example, a classical histological examination, for instance the evaluation of the Gleason grade. However, such a method cannot be conducted in real time due to the preparation processes that are required.

In a particularly advantageous exemplary embodiment, tissue samples 66 that were acquired in the biopsy during the examination c) are therefore likewise supplied to the in vitro analysis unit 34, wherein now tissue-specific cancer markers are measured rather than serum markers. For example, these can be concentration values of RNA (thus gene expression), proteins or metabolites. A molecular diagnosis is thus conducted.

After conclusion of the method according to the invention, a physician 70 can provide a final diagnosis for the patient 2 on the basis of the now complete report sheets 68 and can decide about the further course of treatment.

It is to be noted that this exemplary embodiment concentrates on the diagnosis in order to keep the presentation clear. However, in general the concept of the ProstaStation 30 can also be expanded to therapy approaches or, respectively, integrated theranostic approaches.

In an alternative embodiment of the ProstaStation 30, the already-present data records 82 from entire patient cohorts are drawn upon (along the arrows 80) first to design and later to improve or optimize the decision support system 10 and the analysis software 60. The data records 82 include an integrated medical database 84 in a knowledge management system 86 that contains patient data 88 in the form of data sets. The data sets contain genomic, chemical or image data and laboratory reports, examination results etc., for example. Moreover, the knowledge management system 86 additionally contains a web portal 90.

It is thus possible via data mining, knowledge discovery or other methods of machine learning to extract explicit connections between the data from the method according to the invention and findings from the cohort-centered patient data of the data records 92, to make said connections explicit and to use them for improved analysis of future patients.

In another exemplary embodiment, the knowledge management system 86 is kept in a central location for multiple ProstaStation units 30 so that a larger and more balanced data record 82 is achieved.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method for determining at least one parameter pertaining to the prostate of a patient, comprising the steps of:
in a single integrated diagnostic support system, determining a first risk value for the presence of a prostate illness using a screening method by interaction of the patient with the single integrated diagnostic support system;
when said first characteristic risk value exceeds a limit value, determining at least one second characteristic risk value for the presence of a prostate illness at a suspected location of the prostate illness by generating a medical image of the prostate of the patient by interaction of the integrated diagnostic support system with the patient; and
from said single integrated diagnostic support system, automatically generating a written report describing said first or second characteristic risk values and, if applicable, the location of the prostate illness.

2. A method as claimed in claim 1 wherein said limit value is a first limit value, and comprising, when said second characteristic risk value exceeds a second limit value, obtaining a tissue sample from the prostate of the patient by invasive interaction of the single integrated diagnostic support system with the patient.

3. A method as claimed in claim 1 comprising implementing the screening method with a mini-laboratory analysis unit in said single integrated diagnostic support system.

4. A method as claimed in claim 1 comprising automatically determining said first characteristic risk value using an IT-based decision support system in said single integrated diagnostic support system.

5. A method as claimed in claim 1 comprising determining said second characteristic risk value using prostate-specific variations.

6. A method as claimed in claim 1 comprising determining said second characteristic risk value as a local risk distribution using an automated evaluation system in said single integrated diagnostic support system.

7. A method as claimed in claim 1 comprising implementing said invasive method using said medical image for guidance.

8. A method as claimed in claim 1 comprising automatically determining said first characteristic risk value and said second characteristic risk value using a knowledge management system in said single integrated diagnostic support system.

9. An apparatus for determining at least one parameter pertaining to the prostate of a patient, comprising:
a mini-laboratory analysis unit that interacts with a patient to conduct a prostate screening test on the patient;
an artificially intelligent design support system that automatically determines a first characteristic value for the presence of a prostate illness using said screening test;
a first interface connected to an imaging system that interacts with the patient to produce a medical image of the prostate of the patient;
an automated evaluation system that determines at least one second characteristic risk value for the presence of the prostate illness, and a suspected location of the prostate illness, from the medical image;
a report system having a user interface that emits, via said user interface, a humanly perceptible representation of at least one parameter pertaining to the prostate of the patient indicative of said first or second characteristic risk values and, if applicable, said location of the prostate illness; and
said mini-laboratory analysis unit, said artificially intelligent design support system, said first interface, said imaging system, said automated evaluation system and said automated report system being combined as a single integrated diagnostic support system at one location.

10. An apparatus as claimed in claim 9 comprising a second interface for an invasive prostate examination system to obtain a tissue sample of the patient at said location, said second interface being in said single integrated diagnostic support system.

11. An apparatus as claimed in claim 9 comprising a knowledge management system that generates said at least one parameter, said knowledge management system being in said integrated diagnostic support system.

12. An apparatus as claimed in claim 11 wherein said knowledge management system comprises a medical patient database and an Internet interface.
